(19) United States
(12) Patent Application Publication
(10) Pub. No.: US 2025/0195802 A1
(43) Pub. Date: Jun. 19, 2025

(54) Adjustable Respiratory System of Concentration - Modulable Hydrogen and Oxygen Ventilator

(71) Applicant: NORTH-VISION TECH. INC., Hsinchu (TW)

(72) Inventors: LIN-HSIANG HUANG, Hsinchu (TW); PO-CHIEH HUANG, Hsinchu (TW)

(21) Appl. No.: 18/539,136

(22) Filed: Dec. 13, 2023

(51) Int. Cl. A61M 16/00; A61M 16/20

(52) U.S. Cl. CPC A61M 16/0003; A61M 16/021; A61M 16/204; A61M 2016/0015; A61M 2205/18

(57) ABSTRACT

Disclosed is an adjustable respiratory system of concentration—modulable hydrogen-oxygen ventilator, including: a supplying hydrogen-oxygen mixed gas auxiliary device, a pure water electrolysis hydrogen-oxygen generator including an ion exchange membrane, an oxidation catalyst layer and a reduction catalyst layer; a pair of diffusive metallic layers; an anode conductively connected to the anode metallic layer, and a cathode conductively connected to the cathode metallic layer; a sealed accommodation body being provided with a water inlet, a hydrogen hole, and an oxygen hole; a humidifier bottle including an oxygen delivery tube distal from the oxygen hole, a hydrogen delivery tube distal from the hydrogen hole being respectively inserted into the clean water in humidifier, and a hydrogen-oxygen mixed gas output tube. A hydrogen concentration detector inside or outside the ventilator provides alert and safety for the users and facilities.

### FIELD

The disclosure relates to an adjustable respiratory system, and more particularly relates to a respiratory system of a concentration-modulable hydrogen-oxygen mixed gas generator as a ventilator.

### BACKGROUND

Hydrogen exists on earth substantially in compounds such as water and organic materials. Hydrogen is a colorless, odorless, tasteless, non-metallic gas. Due to its properties such as nontoxicity, zero-radioactivity, easy collection, and reaction with oxygen to release high energy, hydrogen has wide applications in fields of chemistry, physics, engineering, and physiology. Particularly in medical science, as unveiled in “Hydrogen/oxygen therapy for the treatment of an acute exacerbation of chronic obstructive pulmonary disease (COPD): results of a multicenter, randomized, double-blind, parallel-group controlled trial” (Zhang, et al., 2021, 22:149, Respiratory Research), AECOPD (acute exacerbation of chronic obstructive pulmonary disease) patients are currently inhaled with a relatively high concentration of oxygen as a normal treatment; however, comparing to the Control Group who only received oxygen entering onto the plateau period thereafter unable to be improved continuously, the Experimental Group with a hydrogen+oxygen therapy, finding that the patients had a more significant improvement in BCSS score over time (from day 1 to day 7) without a plateau period being observed.

Additionally, Ji-Bing Chen, You-Yong Lu, and Ke-Cheng Xu published “A narrative review of hydrogen oncology: from real world survey to real world evidence” in Medical Gas Research (2020), reporting previous real-world studies on hydrogen oncology, finding that for many cancer patients, dyspnea is effectively ameliorated by adding a portion of hydrogen to the inhaled gas, and over 40% of the patients showed significant improvement of living; some patients were observed with tumor marker decrease, and those patients with nasopharyngeal carcinoma had subsided secretions. The scientists inferred that, by adding hydrogen to the inhaled gas, the antioxidative activity of the hydrogen is expected to react with free radicals of hydroxyl to prevent its injury to our body. A similar finding can be found in Oncology Letters (2020, 20:258).

The studies on respiratory therapies reveal that adding hydrogen to a relatively high oxygen concentration has become a very important direction of treatment in current medical research. Hydrogen can be obtained by ways of for example: thermochemistry, water-gas shift reaction, water electrolysis, steam reforming and etc. Particularly, steam reformer of natural gas is a mass production method to generate hydrogen. When steam and methane react at a high temperature around 1000˜1400-degree K, the hydrogen with carbon monoxide can be produced. In this reaction, the production rate of hydrogen is more efficient under a lower pressure applied to the reaction process. Yet the purification system of hydrogen prefers a higher pressure. In order to compromise and shorten the production time, a high pressure is finally applied in the reaction system. Thus, this system may restrict the production efficiency of hydrogen. Nevertheless, the production process requires elevating both pressure and temperature. But in such condition, the hydrogen molecules may be easily decomposed into hydrogen atoms with high potential energy to react with other elements. If so, this may significantly deteriorate the purity of hydrogen through this production. Moreover, due to its high activity, storing a large volume of hydrogen is always a big concern of Safety.

Another typical hydrogen production process is water electrolyzing, where an electrolyte is added in the pure water to perform an ionic solution. With the direct current passing through a pair of electrodes, the reaction of water electrolysis occurs, where the water is now electrolyzed into hydrogen and oxygen through a redox reaction. However, this method has much safety concern in medical applications because the electrolyte being added is typically strong acid or strong base. But without this electrolyte, the pure water cannot be electrolyzed.

A new technology being developed by using proton exchange membrane, the pure water can be electrolyzed without any electrolyte involved into reaction. In water electrolysis equipment, the pure water enters a reaction chamber from an anode side; besides the electrodes, an anode diffusion layer and an anode catalyst layer with metallic mesh shape are further provided in the reaction chamber. By electrolyzing, the pure water is split into oxygen ions and hydrogen ions via the anode diffusion layer and the catalyst layer, where the oxygen ions are conducted to the anode metallic mesh to release electrons and discharged as oxygen; while the hydrogen ions pass through the proton exchange membrane to a cathode catalyst layer and a cathode diffusive layer; due to electrical conductivity and permeability of the cathode diffusive layer, the electrons supplied from the cathode are received by protons (hydrogen ions) to reduce into hydrogen to be discharged.

The hydrogen and oxygen resulting from the electrolysis described supra are potentially dangerous in a typical environment. With a concentration within the range of 4%˜94% and at a temperature over 287° C., the hydrogen may be ignited and exploded. Since the respiratory therapy is directly applied to the patient, it is required to assure complete safety to the patient as well as the medical facility. Therefore, the application of medical-purpose requires more vigilance and conservative comparing to industrial-purpose, and how to enhance overall safety of use is the top point of this disclosure.

Secondly, with continued real-world studies the patients with individual therapy may have different demands on the mixed ratio of hydrogen and oxygen. Therefore, how to friendly adjust the concentrations of hydrogen and oxygen by the respiratory system is the second point to address by the disclosure.

Detailed features and advantages of this disclosure will be described in the embodiments below, the contents of which would suffice for any person of normal skill to understand and implement the technical contents of this disclosure, and based on this disclosure of the specification, the appended claims, and the drawings, any person of normal skill in the art would easily understand the objectives and advantages of this disclosure.

### SUMMARY

A main objective of this disclosure is to provide an adjustable respiratory system of concentration-modulable hydrogen-oxygen ventilator, which integrates a pure water electrolysis hydrogen-oxygen generator with a humidifier bottle and a hydrogen concentration detector, wherein clean water in the humidifier bottle maintains the hydrogen-oxygen mixed gas at a temperature close to room temperature, effectively preventing abrupt elevation of temperature leading to risk of combustion or explosion; and in further with the hydrogen concentration detector design, the safety risk is effectively lowered.

Another objective of this disclosure is to provide an adjustable respiratory system of concentration-modulable hydrogen-oxygen ventilator, which by vigilantly detecting hydrogen leakage in a surrounding environment via a hydrogen concentration detector embedded or provided inside the room, enables to cut off the power of the pure water electrolysis hydrogen-oxygen generator, thereby suspending continued generation of hydrogen and oxygen to prevent any accidence.

A further objective of this disclosure is to provide an adjustable respiratory system of concentration-modulable hydrogen-oxygen ventilator, which enables this system, by modulating the electric current to control the output efficiency of pure water electrolysis hydrogen-oxygen production, to change the concentration of the gas inhaled by the user.

A still further objective of this disclosure is to provide an adjustable respiratory system of concentration-modulable hydrogen-oxygen ventilator, where nitrogen gas in the air is filtered off via a molecular sieve, and a compressor is employed to control the output efficiency of filtered oxygen into the clean water in humidifier, whereby an oxygen proportion in a mixed gas output is adjusted, and a gas regulating valve is further employed to regulate hydrogen and oxygen proportions to control gas throughput in the regulating tube.

To achieve the objectives stated supra, this disclosure relates to an adjustable respiratory system of concentration-modulable hydrogen-oxygen ventilator, configured to at least change concentration of the gas inhaled by a user, the respiratory system comprising: a supplying hydrogen-oxygen mixed gas auxiliary device configured to supply part of the gas inhaled by the user; a pure water electrolysis hydrogen-oxygen generator performing to split pure water into hydrogen and oxygen output, the pure water electrolysis hydrogen-oxygen generator comprising: at least one ion exchange membrane for ions to pass through, one oxidation catalyst layer and one reduction catalyst layer being coated on two opposite sides of the ion exchange membrane, respectively; a pair of diffusive metallic layers having the plurality of pores, including one anode metallic layer closely adjacent to the oxidation catalyst layer, and the other cathode metallic layer closely adjacent to the reduction catalyst layer; at least one pair of electrodes, including an anode conductively connected to the anode metallic layer, and a cathode conductively connected to the cathode metallic layer; and a sealed accommodation body for accommodating the ion exchange membrane, the diffusive metallic layers, and the electrodes, the sealed accommodation body being provided with a water inlet, a hydrogen hole, and an oxygen hole, the deionized water being able to inject into the sealed accommodation body through the water inlet; a humidifier bottle, comprising an oxygen delivery tube connected to the oxygen hole, a hydrogen delivery tube connected to the hydrogen hole, a humidified mixed gas connected to the supplying hydrogen-oxygen mixed gas auxiliary device, and the humidifier bottle holding clean water, wherein one end of the oxygen delivery tube distal from the oxygen hole and the other end of the hydrogen delivery tube distal from the hydrogen hole from the accommodation body, respectively, and another end of the humidified mixed gas output tube positioned higher than the clean water, supply the hydrogen-oxygen mixed gas to the auxiliary device; and a hydrogen concentration detector configured to detect hydrogen concentration can output an alarm signal when the hydrogen concentration exceeds a predetermined standard or limit.

In this disclosure, by integrating the humidifier bottle and the hydrogen concentration detector, the pure water electrolysis hydrogen-oxygen generator enables, on one hand, the outputted hydrogen and oxygen mixed gas maintaining at a constant temperature close to room temperature, which effectively prevents a too high operating temperature causing a potential gas explosion or combustion risk and prevents safety hazards to the user, and also enables detection, with a hydrogen concentration detector, of whether hydrogen leaks causing abnormal change of the surrounding. With an alarm signal, the system can cut off the power to the pure water electrolysis hydrogen-oxygen generator to suspend the continued generation of the hydrogen and oxygen. Besides, by incorporating a molecular sieve, more oxygen can be provided, and the oxygen from molecular sieve is introduced into the clean water of humidifier bottle, whereby the oxygen proportion can be adjusted by the compressor of molecular sieve, and the portions of hydrogen-oxygen as well as gas throughput can be adjusted by using a gas regulating valve.

### BRIEF DESCRIPTION OF THE DRAWINGS

FIG. 1 is a structural schematic diagram of a pure water electrolysis hydrogen-oxygen generator integrated with a supplying hydrogen-oxygen mixed gas auxiliary device, a humidifier bottle, and a hydrogen concentration detector according to the disclosure.

FIG. 2 is an exploded view of an internal structure of a pure water electrolysis hydrogen-oxygen generator according to the disclosure.

FIG. 3 is a sectional view of an internal structural configuration of a sealed accommodation body illustrated in FIG. 1.

FIG. 4 is a structural schematic diagram of the pure water electrolysis hydrogen-oxygen generator with molecular sieve, which provide adjustability of gas-throughput according to the disclosure.

FIG. 5 is a structural schematic diagram of two sets of pure water electrolysis hydrogen-oxygen generator mechanisms provided inside the sealed accommodation body.

FIG. 6 is an application status schematic diagram of a hydrogen concentration detector mounted at the ceiling in a room.

FIG. 7 is a schematic diagram of pure water electrolysis via an anion exchange membrane.

### DETAILED DESCRIPTION OF EMBODIMENTS

Hereinafter, embodiments of this disclosure will be illustrated via specific examples and those skilled expertise in the art may easily understand other benefits and effects of the disclosure via depictions in the specification.

The structures, proportions, and sizes illustrated in the drawings are only intended for facilitating those skilled in the art to understand and read the illustration or specification, but not intended to limit the disclosure; therefore, they do not have the substantive meanings in technical; any structural modifications, proportional relationship alterations, or size adjustments shall fall within the scope of the disclosure without affecting the effect generated or objective achieved by this disclosure. Meanwhile, the terms such as “one”, “two”, or “above” referred to herein only serve to case the description, but not be intended for limiting the scope implementable by the disclosure. And the change or adjustment of corresponding relative relationships shall be regarded as falling into the scope of the disclosure without substantive change of the contents in technical.

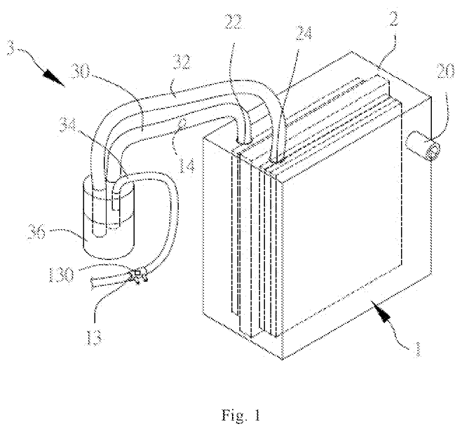

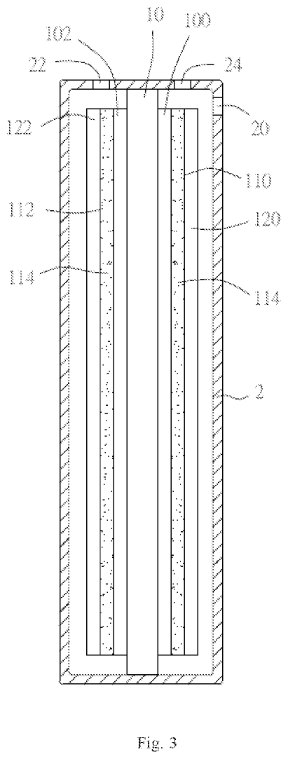

FIGS. 1, 2, and 3 illustrate a pure water electrolysis hydrogen-oxygen generator 1 according to the first example embodiment of this disclosure. The pure water electrolysis hydrogen-oxygen generator 1 is accommodated in a sealed accommodation body 2, the sealed accommodation body 2 being provided with a water inlet 20, a hydrogen bole 22, and an oxygen hole 24; and deionized water, which is injected into the sealed accommodation body 2 via the water inlet 20, is enclosed by the sealed accommodation body 2 and connected to the oxygen bole 24 and the hydrogen hole 22.

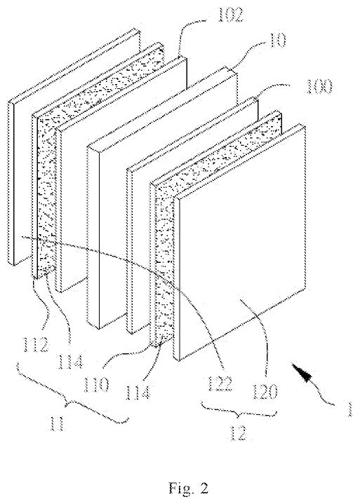

By referring to FIG. 2, an ion exchange membrane 10 in the pure water electrolysis hydrogen-oxygen generator 1 is set as a symmetrical centerline. An oxidation catalyst layer 100 and a reduction catalyst layer 102 are coated on two opposite sides of the ion exchange membrane 10, respectively; the diffusive metallic layers 11 are composed by an anode metallic layer 110 which is just close to the oxidation catalyst layer 100 and a cathode metallic layer 112 which is just close to the reduction catalyst layer 102. Each diffusive metallic layer is provided with plurality of pores 114, so that after the hydrogen ions or oxygen ions are oxidized or reduced respectively, the generated hydrogen molecules or oxygen molecules can be easily separated from water to discharge, whereby the gas generation efficiency is enhanced. The electrodes 12 compose an anode electrode 120 conductively connected to the anode metallic layer 110, and a cathode electrode 122 conductively connected to the cathode metallic layer 112.

Furthermore, a humidifier bottle 3 is additionally provided, comprising a hydrogen delivery tube 30 inserted in the hydrogen hole 22, an oxygen delivery tube 32 inserted in the oxygen bole 24, and a humidified mixed gas output tube 34 delivered to a supplying hydrogen-oxygen mixed gas auxiliary device 13 (e.g., a nasal cannula). One end of the oxygen delivery tube 32 distal from the oxygen hole 24 and another end of the hydrogen delivery tube 30 distal from the bydrogen hole 22, are respectively inserted into the bottle body 36 and submerged into the clean water filled in the bottle body 36; the other end portion of the humidified mixed gas output tube 34 distal from the supplying hydrogen-oxygen mixed gas auxiliary device 13 is also inserted into the bottle body 36 but positioned higher than the clean water.

In operation, deionized water is injected via the water inlet 20 of the sealed accommodation body 2 till the liquid level is close to the oxygen hole 24 or the hydrogen hole 22; then, a switch is turned on to energize the electrode 12, starting the electrolysis process. A space for the deionized water to circulate is reserved through a plurality of pores of diffusive metallic layer 110 to the oxidation catalyst layer 100 as well as a plurality of pores of diffusive metallic layer 112 to the reduction catalyst layer 102, respectively. When the deionized water accesses the metallic layer 110 connected to the anode 120, positively charged hydrogen ions or negatively charged hydroxide ions are released through the ion exchange membrane 10 from the side of catalyst layer 100 to the other side of catalyst layer 102. In this example, the ion exchange membrane is a proton exchange membrane, which allows hydrogen ions to pass through proton exchange membrane 10 to thereby effectuate current conduction and the redox reaction.

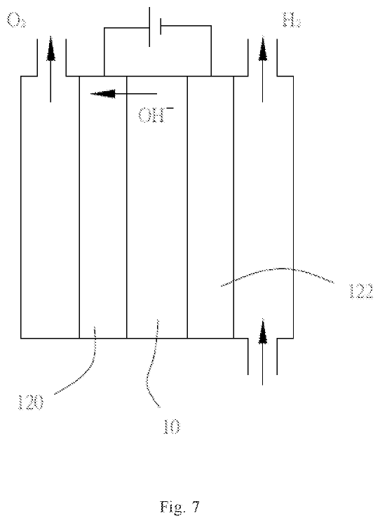

Furthermore, the hydrogen ions pass through the ion exchange membrane 10 and the reduction catalyst layer 102 to access the diffusive metallic layer 11 composing a plurality of pores 114 of the cathode metallic layer 112, so that the cathode 122 extensively releases electrons, which are bonded with the hydrogen ions to form hydrogen molecules. Instead of proton exchange membrane, if the ion exchange membrane is an anion exchange membrane, by referring to FIG. 7, the hydroxide ions may pass through ion exchange membrane 10 to access the anode metallic layer 110 connected to the anode 120 to release electrons and form the oxygen molecules. Since the electrolysis process uses deionized pure water and is thus free of any strong acid or strong base, hydrogen of higher purity may be efficiently obtained; the hydrogen is then discharged into the clean water via the hydrogen delivery tube 30, while the oxygen is discharged into the clean water via the oxygen delivery tube 32, where the hydrogen and the oxygen are mixed into a desired hydrogen-oxygen mixed gas; since the temperature of the clean water is maintained at a fixed normal temperature appropriate for the human body, the hydrogen and oxygen can be limited to a safe temperature range far lower than the combustible temperature, whereby any risk of abruptly elevated temperature causing explosion or combustion to endanger the users or medical facilities can be prevented; the hydrogen-oxygen mixed gas is discharged via the humidified mixed gas output tube 34 positioned higher than the clean water within bottle body 36, and then supplied, via the supplying hydrogen-oxygen mixed gas auxiliary device 13, to the user for inhalation.

Since this example does not employ a mask to supply the mixed gas, concentration of the gas doped into indoor air and applied to the user generally comprises about 22% of oxygen and about 4% of hydrogen, with the rest of nitrogen. Of course, if the patient has a special need, an open type of nasal cannula may not be employed here; instead, the gas is supplied otherwise with stricter indoor air control and raised the volume as well as accuracy of hydrogen-oxygen proportions.

To control hydrogen never leaked from the sealed accommodation body, a hydrogen concentration detector 14 (a SnO₂ resistor) is disposed at a side close to the hydrogen hole 22 of the sealed accommodation body, configured to output an alarm signal while the hydrogen concentration exceeding a predetermined standard or limit.

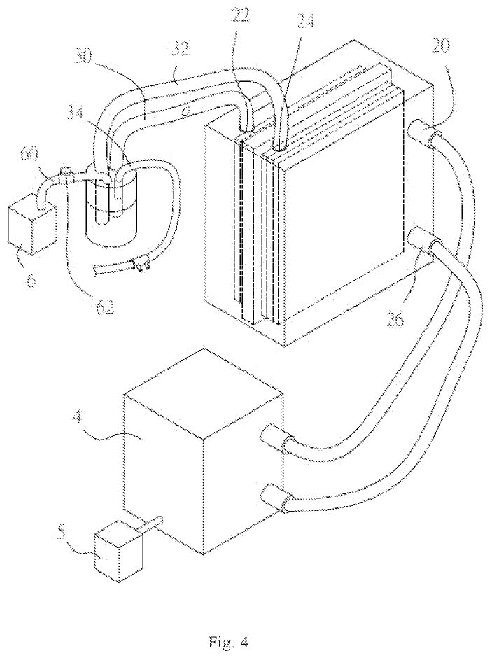

Moreover, refer to FIG. 4 in combination, which is a structural schematic diagram of the pure water electrolysis hydrogen-oxygen generator with gas-throughput adjustability according to the disclosure, where the pure water electrolysis hydrogen-oxygen generator 1 is further optimized in terms of heat dissipation effect and adjustable hydrogen and oxygen proportions. For optimizing the heat dissipation effect, a water outlet 26 is provided from the sealed accommodation body 2, and a circulating water tank 4 is also provided. The circulating water tank 4 is connected to the water inlet 20 and the water outlet 26, thereby reaching a thermal balance between the sealed accommodation body 2 and the circulating water tank 4; to enable the circulating water tank 4 to maintain a stable temperature status, the circulating water tank 4 is additionally thermally connectable to a heat sink 5 configured to conduct heat out to stabilize the temperature of the water within the circulating water tank 4 and the sealed accommodation body 2.

Moreover, for adjustable hydrogen and oxygen proportions, a molecular sieve 6 may be employed to enhance oxygen proportion; in some specific implementations, the molecular sieve 6 is configured to receive the oxygen by compressing the air with filtering the nitrogen. The oxygen by the molecular sieve 6 is delivered through an oxygen proportion regulating tube 60 being introduced into the clean water in bottle body 36, thereby adjusting oxygen proportion in the humidified mixed gas output tube 34. The gas throughput of the oxygen proportion regulating tube 60 is adjusted via a gas regulating valve 62. In a specific operation, the supplying hydrogen-oxygen mixed gas auxiliary device 13 is worn by a user; the original hydrogen-oxygen mixed gas passing through the clean water would be humidified, and then the humidified hydrogen-oxygen mixed gas is delivered via the humidified mixed gas output tube 34 to the supplying hydrogen-oxygen mixed gas auxiliary device 13 so as to be supplied to the user. When the user needs to increase the oxygen content, the amount of oxygen outputted in the clean water may be adjusted via a gas regulating valve 62; at this point, the oxygen content in the original hydrogen-oxygen mixed gas increases in equal proportion to adjustment by the gas regulating valve 62. In addition, for the pure water electrolysis generator part, production efficiency of hydrogen and oxygen from the pure water electrolysis may be enhanced by controlling the electrolytic current. Since the amount of oxygen production from the molecular sieve is generally greater than that from the pure water electrolysis hydrogen-oxygen generator, controlling the electrolytic current of the pure water electrolysis hydrogen-oxygen generator mainly serves as modulating the proportion of hydrogen production. In this way, the modulated hydrogen-oxygen mixed gas delivered from the humidified mixed gas output tube 34 to the supplying hydrogen-oxygen mixed gas auxiliary device 13 is adjustable in hydrogen and oxygen proportions respectively. Meanwhile, by the gas concentration detector 130 disposed in the supplying hydrogen-oxygen mixed gas auxiliary device 13 (i.e., nasal cannula), the real values of the hydrogen and oxygen proportions can be immediately obtained and can be timely adjusted operated as supra to fit a predetermined proportions of hydrogen and oxygen for different purposes of medical use. This can also prevent the user from feeling uncomfortable or having an adverse reaction.

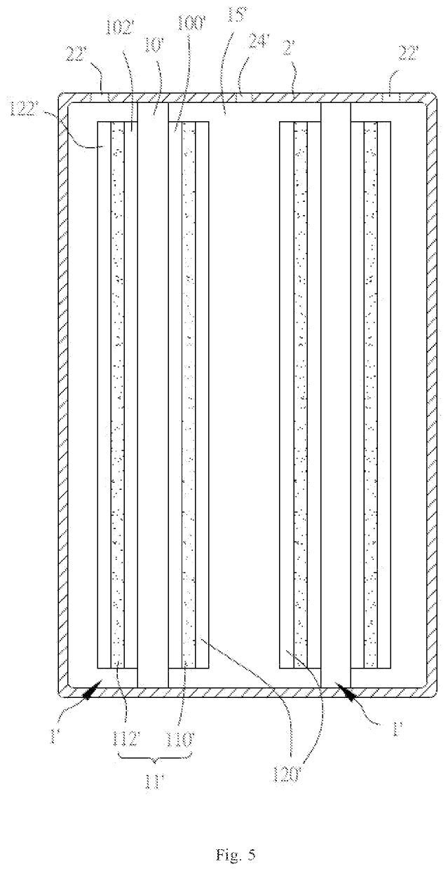

Refer to FIG. 5 in combination, which is a structural schematic diagram of two sets of pure water electrolysis hydrogen-oxygen generators provided inside the sealed accommodation body according to a second example embodiment of this disclosure. The two sets of pure water electrolysis hydrogen-oxygen generators 1′ with an ion exchange membrane as a center are accommodated in the sealed accommodation body 2′; an oxidation catalyst layer 100′ and a reduction catalyst layer 102′ are coated to the two sides of the ion exchange membrane 10′ in each individual pure water electrolysis hydrogen-oxygen generator 1′, respectively; the diffusive metallic layers 11′ are composed by an anode metallic layer 110′ which is just close to the oxidation catalyst layer 100′ and a cathode metallic layer 112′ which is just close to the reduction catalyst layer 102′. The anode electrode 120′ is conductively connected to the anode metallic layer 110′, and the cathode electrode 122′ is conductively connected to the cathode metallic layer 112′. The individual pure water electrolysis hydrogen-oxygen generators 1′ are configured in a mirrored set, so that the respective ion exchange membranes hold a common water tank 15′. And the common oxygen hole 24′ is located at top of water tank 15′ of the sealed accommodation body 2′, and the hydrogen holes 22′ are separately located at top of two sides of the sealed accommodation body 2′.

As noted supra, the production efficiency of hydrogen and oxygen is varied with increase by the sealed accommodation body 2′. By controlling the electrolysis current, the production efficiency of hydrogen and oxygen can be controlled. Meanwhile, provision of the molecular sieve may effectively enhance the oxygen content.

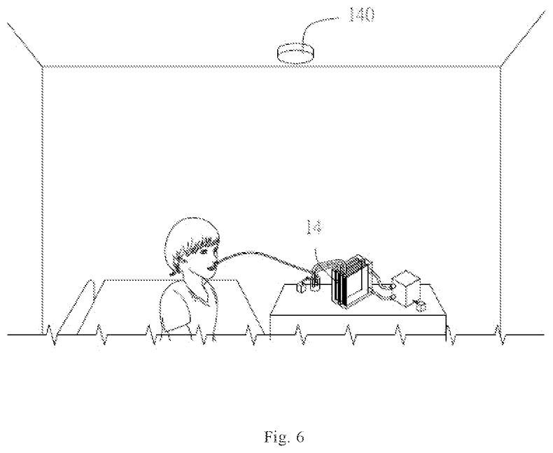

Refer to FIG. 6 in combination, which is an application status schematic diagram of a hydrogen concentration detector provided at the ceiling in a room. Irrespective of which implementation patterns described supra, when being applied inside a room, instead of the hydrogen concentration detector 14, a sensing element can be additionally provided with at the ceiling position. As hydrogen is colorless, odorless, and highly combustible substance with a density far less than atmosphere density under a typical atmospheric pressure, leaked hydrogen would be accumulated at the upper air, and if the room is poorly ventilated, hydrogen concentration would increase, which is easily ignited and exploded by external factors; therefore, the sensing element 140 mounted on the ceiling may detect the hydrogen concentration status at the first time and may generate an alarm signal to respiratory system when the hydrogen concentration exceeds the threshold, to cut off both powers to the pure water electrolysis hydrogen-oxygen generator and to the molecular sieve to stop continuous generation of hydrogen and oxygen. Of course, the hydrogen concentration detector is not limited only to being disposed at the ceiling, which may also be disposed inside the respiratory system to detect whether hydrogen leaks and issue an alarm to shut down the system. Furthermore, those skilled in the art can easily understand that the alarm system of hydrogen leakage is not limited to the hydrogen concentration detector. A pressure detector can be also adopted to connect at the hydrogen delivery tube or the oxygen delivery tube, respectively, to detect in-tube pressure change to alert whether the hydrogen delivery tube or the oxygen delivery tube leaks, which may also provide safety.

FIG. 7 shows a third example embodiment of this disclosure. Since the ion exchange membrane is not limited only to a proton exchange membrane allowing hydrogen ions to pass through, which may also be an anion exchange membrane allowing hydroxide ions to pass through; therefore, the hydroxide ions will pass through the exchange membrane to be thereby electrically charged in this example.

The example embodiments described supra are only intended to exemplarily illustrate the principle and effect of the disclosure, not intended to limit the disclosure. Any person of normal skill in the art may modify the example embodiments without departing from the spirit and scope of the disclosure. Therefore, the scope sought for protection by this disclosure shall be limited in the appended claims.

I/We claim:

1. an adjustable respiratory system of concentration-modulable hydrogen-oxygen ventilator, configured to at least change concentration of a gas inhaled by a user, the respiratory system comprising:
a supplying hydrogen-oxygen mixed gas auxiliary device configured to supply part of the gas inhaled by the user;
a pure water electrolysis hydrogen-oxygen generator configured to split pure water into hydrogen and oxygen to output, the pure water electrolysis hydrogen-oxygen generator comprising:
at least one ion exchange membrane for ions to pass through, one oxidation catalyst layer and one reduction catalyst layer being coated on two opposite sides of the ion exchange membrane, respectively;
a pair of diffusive metallic layers having a plurality of pores, including one anode metallic layer disposed just close to the oxidation catalyst layer, and the other cathode metallic layer disposed just close to the reduction catalyst layer;
at least one pair of electrodes, including an anode conductively connected to the anode metallic layer, and a cathode conductively connected to the cathode metallic layer; and
a sealed accommodation body for accommodating the ion exchange membrane, the diffusive metallic layers, and the electrodes, the sealed accommodation body being provided with a water inlet, a hydrogen hole, and an oxygen hole, deionized water injected into the sealed accommodation body via the water inlet being enclosed by the sealed accommodation body and connected to the oxygen hole and the hydrogen hole;
a humidifier bottle, comprising an oxygen delivery tube connected to the oxygen hole, a hydrogen delivery tube connected to the hydrogen hole, a humidified mixed gas output tube connected to the supplying hydrogen-oxygen mixed gas auxiliary device, and a bottle body holding clean water, wherein one end of the oxygen delivery tube distal from the oxygen hole and one end of the hydrogen delivery tube distal from the hydrogen hole are inserted into the bottle body and submerged in the clean water, respectively, and an end portion of the humidified mixed gas output tube distal from the supplying hydrogen-oxygen mixed gas auxiliary device is positioned higher than the clean water in the bottle body; and
a hydrogen concentration detector configured to detect hydrogen concentration close to the sealed accommodation body to output an alarm signal when the hydrogen concentration exceeds a predetermined limit.

2. The respiratory system according to claim 1, wherein the hydrogen concentration detector comprises a tin oxide (SnO₂) resistor.

3. The respiratory system according to claim 1, wherein the respiratory system is disposed in a room and configured to be used by the user in the room, and the hydrogen concentration detector further comprises a sensing element disposed at a ceiling corresponding to the room.

4. The respiratory system according to claim 1, wherein the pure water electrolysis hydrogen-oxygen generator comprises two ion exchange membranes, two anode metallic layers which are proximal to each other, and two cathode metallic layers which are distant from each other, an anode common water tank being held between two sheets of ion exchange membrane, the ion exchange membrane being a proton exchange membrane for hydrogen ions to pass through or an anion exchange membrane for hydroxide to pass through.

5. The respiratory system according to claim 1, wherein the sealed accommodation body further comprises a water outlet, and the respiratory system further comprises a circulating water tank connected to the water inlet and the water outlet, whereby thermal balance between the temperature in the sealed accommodation body and the circulating water tank is assured.

6. The respiratory system according to claim 5, wherein the circulating water tank is further thermally conductively connected to a heat sink.

7. The respiratory system according to claim 1, further comprising an alarm handler configured to receive the alarm signal and cut off power to the pure water electrolysis hydrogen-oxygen generator, thereby stopping continued generation of hydrogen and oxygen.

8. The respiratory system according to claim 1, further comprising a molecular sieve configured to provide more oxygen by filtering nitrogen from the air, the molecular sieve being further connected to an oxygen proportion regulating tube configured to introduce oxygen molecules into the clean water of bottle body, whereby oxygen percentage in the humidified mixed gas output tube is adjustably increased.

9. The respiratory system according to claim 8, further comprising a gas regulating valve configured to adjust a gas throughput of the oxygen proportion regulating tube.

10. The respiratory system according to claim 9, wherein the supplying hydrogen-oxygen mixed gas auxiliary device comprises a nasal cannula and a hydrogen and oxygen concentration detecting element disposed corresponding to the nasal cannula.
